(19) United States
(12) Patent Application Publication
(10) Pub. No.: US 2016/0192877 A1
(43) Pub. Date: Jul. 7, 2016

(54) GUIDING/SUPPORTING STRUCTURE FOR COUPLING TO A LIVING BEING, AND METHOD FOR DETERMINING SUITABLE CONTACT SURFACES ON THE LIVING BEING

(71) Applicant: EXOS GMBH, München (DE)

(72) Inventors: Laura DIEZ, Kempten (DE); Volker JUNIOR, Graefelfing (DE); Greta STEHLING, Thansau (DE)

(73) Assignee: EXOS GMBH, München (DE)

(21) Appl. No.: 14/654,785

(22) PCT Filed: Dec. 19, 2013
(86) PCT No.: PCT/EP2013/077460

§ 371 Date: Jun. 22, 2015

(30) Foreign Application Priority Data
Dec. 21, 2012 (DE) 10 2012 025 431.8

(51) Int. Cl. A61B 5/00; A61B 5/107; A61F 2/50; A61F 5/01; A61F 2/78

(52) U.S. Cl. CPC A61B 5/4836; A61F 5/0102; A61F 2/78; A61F 2/5046; A61B 5/1077; A61B 5/7246; A61B 5/742; A61B 2034/105

(57) ABSTRACT

The present invention relates to a guiding/supporting structure and to a method for detecting surface deformations of body surfaces of living beings for determining suitable contact surfaces for guiding/supporting structures, which can be brought into contact with the surfaces of the living beings. The method comprises:
orienting a body part of the living being with respect to a detection device for at least partially detecting the surface shape of at least one surface area of the body part,
detecting a first surface characteristic of the surface area when the body part is in a first load state, in particular a state with the muscles relaxed,
detecting a second surface characteristic of the surface area when the body part is in a second load state, in particular a state with the muscles tensioned, and
determining the local surface deformations resulting between the first load state and the second load state.

The present invention relates to a method for detecting surface deformations of body surfaces of living beings for determining suitable contact surfaces for guiding/supporting structures, which can be brought into contact with the surface of the living beings, in particular orthoses, as well as to a guiding/supporting structure for coupling to a living being.

The coupling of guiding/supporting structures to living beings, in particular to humans, requires the consideration of a variety of thoughts. In contrast to the application of guiding/supporting structures to mechanical devices or to buildings, e.g., bodies of living beings always differ from one another. This results, e.g., from different body sizes, from different muscle and tissue structures, from different length ratios of the legs to the upper part of the body and from many more. In contrast to a connection of a guiding/supporting structure to a mechanical device, an enormous complexity already results from this in a readily identifiable manner.

It is furthermore important to consider that living beings feel pain and that the contact locations, to which a guiding/supporting structure is attached or can be attached, respectively, can become sore. The guiding/supporting structures also need to be designed such that they do not cause any constrictions of body regions, which would lead to pain. Nonetheless, the guiding/supporting structure should not slip at the coupled body region, which is often criticized as being very bothersome and whereby the functionality of a guiding/supporting structure is at least limited or reduced. Furthermore, a guiding/supporting structure is not to cause an unintentional impediment of the movements of the person, if possible.

Publication DE102005008605A1 discloses a method for creating an external prosthesis or orthosis. A tomography of a body part is generated, the thereby generated data are converted into a 3-D model, the bones, muscles and fatty tissue structure is evaluated, compression zones are determined as a function of the evaluation and a prosthesis/orthosis element is generated as a function of this data. DE102005008605A1 thereby considers the knowledge that, in the event of amputations, individual muscles lose their attachment and that the muscle mass is reduced and is replaced by fat mass over time, for the design of prostheses. According to DE102005008605A1, the inner structure of a body part is thus analyzed. The prosthesis is connected to the body via a connection across a surface, which is as large as possible. The surface elements used for the connection are thereby designed according to ruled geometries.

Publication EP 1 843 291 A1 also discloses a method, according to which the portions of bone, muscle and fatty tissue are separated by means of a plurality of cross section images of an extremity stump.

The methods known from the state of the art have the disadvantage that they are essentially only suitable for producing prostheses or orthoses, respectively, which are coupled to body parts, in which no muscle activity or only a slight muscle activity can be detected. Transferring these methods or orthoses resulting therefrom, respectively, to body parts with normal muscle activity would lead to an extremely high contact pressure within the surrounding orthosis in response to a tensioning of the skeletal muscles and in response to a local volume increase of the muscle resulting therefrom. This increase of pressure would result in constrictions of the body part and pain or loss or performance, respectively, resulting therefrom or to undesired movement limitations, respectively, for the person wearing such an orthosis.

It is thus the object of the present invention to provide a method and a corresponding guiding/supporting structure, which can be coupled to a body part of a living being, and which provides for the best possible functionality, in particular a high wearing comfort, in particular with considerably fewer constrictions than in the case of the prostheses and orthoses known from the state of the art in response to a normal muscle activity in the body part.

According to the subject matter of claim 1, the above-mentioned object is solved by means of a method for detecting surface deformations of body surfaces of living beings for determining suitable contact surfaces for guiding/supporting structures, which can be brought into contact with the surface of the living beings. Human beings, in particular children, grown-ups, seniors and/or disabled persons and/or injured persons and/or athletes, and/or animals, in particular domestic animals, such as dogs and/or cats, or working animals, such as horses, cows and/or pigs, are hereby preferably understood as living beings.

Such a method comprises at least the steps: orienting at least one body part of the living being with respect to a detection device for at least partially detecting the surface deformations of at least one surface area of the body part, detecting a first surface characteristic or shape, respectively, of the surface area by means of the detection device, wherein the body part is in a first load state, in particular a state with the muscles relaxed and/or in a state with body parts oriented relative to one another in a neutral position, detecting a second surface characteristic or shape, respectively, of the surface area by means of the detection device, wherein the body part is in a second load state, which differs from the first load state, in particular a state with the muscles tensioned and/or in a state with body parts, which are inclined and/or rotated and/or pivoted to one another, and determining the local surface deformations resulting between the first load state and the second load state, in particular by means of an evaluation of the detected surface characteristics or shapes, respectively, or by comparing the detected surface shapes, respectively, in particular by comparing the first surface shape to the second surface shape.

Among others, a guiding/supporting structure can preferably be understood concretely as an orthosis, in particular, arm, knee, joint, leg, hip, shoulder, torso, neck, nape, hand, foot orthosis, a prosthesis, an exoskeleton, a carrier system, in particular a backpack, a bag, a climbing harness, a lifting system, a backpack, a walking aid, in particular crutches, a shoe, in particular a ski boot, a protective device, such as, e.g., a brace, in particular for use in sports, or a protector, in particular to avoid work injuries and/or sports injuries, a stabilizing device, such as a splint for splinting bone fractures, etc., and/or as a combination thereof.

Preferably, one identical device or, particularly preferably, a plurality of identical devices or devices, which differ from one another, can be identified as detection device. Particularly preferably, the detection of the surface deformations or of varying body part volumes, respectively, takes place by means of sensor devices, which can be coupled to the body part or to the surface areas, respectively, and which are embodied in particular for detecting pressure, and/or by means of an optical detection device, in particular an image capturing device or a video capturing device, and/or by means of an emitting device for emitting beams, which permeate the body part, and/or a mechanical and/or fluidic detection device and/or a terahertz scanner. Wherein the sensor devices can preferably be attached flat around the body part, in particular to a pants-like means, and wherein the image capturing device is preferably a 3-D scanner and wherein the emitting device is preferably an X-ray device, a computer tomograph or a magnetic resonance tomograph.

Preferably, the orienting of a body part with respect to a detection device is to be understood as introducing the body part into the effective area of the detection device and/or as transferring the body part into a concrete position with respect to the detection device.

Detecting surface deformations at least in sections preferably describes that not all surface deformations of a body part need to be detected, wherein particularly preferably all surface deformations of a body part are detected.

The term surface characteristic hereby preferably describes the surface structure or the topography, respectively, of the surface, in particular of the skin and/or of the coat of the living being. Particularly preferably, this is to be understood macroscopically, that is, the characteristic or structure, respectively, or shape, which results from deformations, in particular expansions and contractions, e.g. of more than 1 mm and preferably of more than 3 mm and particularly preferably of more than 5 mm.

This method is advantageous, because surface areas, which experience significantly slighter or no deformations in spite of normal muscle activity and even in response to sports activities as compared to other surface areas of the same body part, can be identified by determining the local surface deformations. Guiding/supporting structures can be coupled to these surface areas, which are deformed slightly or which are undeformed, in an advantageous manner, because the risk of constrictions is greatly reduced due to the significantly slighter expansions. The guiding/supporting structures can thus be coupled to the respective body part with significantly lower forces. Furthermore, by attaching a guiding/supporting structure to the slightly deformed or undeformed surface areas, respectively, a shifting of the guiding/supporting structure is virtually impossible, whereby the preserving of the mobility is ensured in an advantageous manner and/or the intended or defined, respectively, or provided, respectively, limitation of mobility is ensured.

Preferably, the method thus serves to determine surface areas of a body part of a living being, which are suitable for being brought into contact with guiding/supporting structures for unidirectional or bidirectional transfer of forces and/or moments, wherein the surface areas are preferably characterized in that, as compared to other surface areas of the same body part, they have slighter deformations between two load states or for determining surface portions, which act statically or substantially statically, respectively, between different load states, respectively, or for determining, which degree of deformation occurs at which position of the detected surface area with respect to an initial state, respectively.

The method can preferably be taken over or adapted to support the production of a guiding/supporting structure and particularly preferably for a production method for producing guiding/supporting structures.

According to a preferred embodiment of the present invention, a comparison of the detected local surface deformations is carried out with at least one defined threshold value or a defined threshold value function, wherein, on the one hand, deformation values resulting therefrom, in particular below the defined threshold value or the defined threshold value function, can be described, defined and/or determined as values having a slight surface deformation, in particular slight expansion or contraction, and, on the other hand, in particular above the defined threshold value or of the defined threshold value function, can be described, defined and/or determined as values of a high surface deformation.

It is possible hereby that provision is not only made for a threshold value and/or a threshold value function for identifying areas of slight and high deformation, but for various degrees of dynamic or static, respectively, to be identifiable in multiple stages, in particular in three, four, five stages, in particular more than five stages. All areas, the deformation of which is below a first threshold value, e.g., can thus be identified as being static, all areas, the deformations of which lie above the first threshold value but below a second threshold value, can be identified as being partially dynamic, and all areas, the deformations of which lie above the second threshold value, can be identified as being dynamic. The different areas can be identified specifically, e.g. for the optical output, they can in particular be dyed according to a color scheme, which is assigned to the respective degree of deformation.

This embodiment is advantageous, because the determined results can be output in an optically prepared and thus in an easily comprehensible manner, e.g. for a person carrying out or accompanying the method, respectively, and/or for documentation purposes, by means of the threshold value or the threshold values, respectively, and/or the threshold value function or the threshold value functions, respectively. In addition, it is possible for a processor device to generate a view, in particular a 3-D view of the body part and/or of the guiding/supporting structure, and/or a production plan and/or the production of a guiding/supporting structure to be initiated or converted, respectively, or to be derived directly in an automated manner by means of the optimal coupling areas, which are determined by means of the threshold values and/or threshold value functions.

The determination of the local surface deformations and/or the comparison of the detected surface deformations is effected according to a further preferred embodiment by means of a processor device. The processor device can thereby be a part of the detection device, e.g. and/or can be coupled or can be capable of being coupled, respectively, to the detection device.

Preferably, the determination and/or the comparison takes place by means of the processor device in a fully automatic or semiautomatic manner.

The processor device preferably comprises a storage device for storing raw data and/or processed data, wherein it is also possible for the storage device and the processor device to only be connected to one another in terms of signaling, that is, for exchanging data. In addition or in the alternative, the processor device is preferably connected to an output device, such as a screen, a printer, an optical drive, a directly controllable production device, in particular a 3-D printer, etc. Furthermore, the processor device is preferably equipped with a processing means or has access to such a processing means, respectively, in particular a computer-aided design tool, by means of which the deformation values can be transferred into data or data file formats, respectively, for the construction or design, respectively, of a supporting/guiding structure.

According to a further preferred embodiment, at least three surface areas are assigned to zones of different surface deformations as a function of at least two defined threshold values or threshold value functions, in particular the surface deformations in the first zone, which can be assigned to the first surface area, are slighter than the surface deformations in the second zone, which can be assigned to the second surface area, and are slighter than the surface deformations in the third zone, which can be assigned to the third surface area, wherein the surface deformations in the third zone with reference to a center of the body part preferably take place in opposite direction to the surface deformations in the second zone. However, it is also possible for the surface deformations in the first zone, which can be assigned to the first surface area, to be larger than the surface deformations in the second zone, which can be assigned to the second surface area, and to be slighter than the surface deformations in the third zone, which can be assigned to the third surface area.

According to a further preferred embodiment of the present invention, an illustration, which corresponds to the deformation values and/or to the surface areas, which can be assigned to the one side of the threshold value or to the threshold value function, is generated and/or visualized for displaying different degrees of deformation at different positions of the surface area, wherein particularly preferably the deformation values and/or the surface areas, which can be assigned to the one side of the threshold value or to the threshold value function, are converted or transferred, respectively, into data for generating a 3-D model, in particular of a body part and/or of a supporting/guiding structure, which is adapted to the body part.

This embodiment is advantageous, because the determined results of a person carrying out or accompanying the method, respectively, and/or for documentation purposes are prepared optically and can thus be output in an easily comprehensible manner. By means of the optical preparation, a verification of the output deformation values can take place directly, in particular by means of a person operating the detection device.

The present invention can furthermore refer to a computer program product, which comprises a program code, which is stored on a computer-readable medium, for carrying out at least individual method steps, which are required for detecting the deformation values and/or the processing thereof and which were described above or which will be described below, respectively, when the computer program product is run on a computer, or refers to a computer-implemented method according to one of claims 1 to 5, respectively. However, it is also possible for the present invention to refer to a control means or to a control software, respectively, in particular for controlling the detection device and/or the processor device and/or a production device.

In addition, the above-mentioned object of the invention is also solved by means of a guiding/supporting structure, in particular an orthosis, for coupling to a living being and for transferring forces, which can be introduced into a first portion of the guiding/supporting structure, to a second portion of the guiding/supporting structure, which is spaced apart from the first portion. The first portion of the guiding/supporting structure is embodied for resting against the surface of a body part of a living being, at least indirectly and at least in sections, and the surface of the body part can be overlaid by the flat section in a contacting manner at least in sections. At least the flat section of the first portion is furthermore embodied for resting against the body part such that, in a defined orientation with respect to the body part, which is provided for fulfilling the function of the guiding/supporting structure, it can be placed against surface areas, which, when transitioning from a first load state into a second load state, encompass surface deformations, which, during the same transition, are slighter than surface deformations of further surface areas of the body part, or, in a defined orientation with respect to the body part, which is provided for fulfilling the function of the guiding/supporting structure, it can be placed against surface areas, which, when transitioning from a first load state into a second load state, encompass surface deformations, which, during the same transition, are higher than surface deformations of further surface areas of the body part.

The flat section preferably encompasses a plurality of, in particular exactly two, two or more than two, in particular exactly three, three or more than three, in particular exactly four, four or more than four areas, by means of which the guiding/supporting structure can preferably be coupled via form closure to the body part or to the body parts, respectively.

Furthermore, the first portion is preferably not only embodied for resting flat against the surface of a body part indirectly and in sections, but can also rest against the body directly, wherein it is also possible for the first portion to not only rest flat against the surface of the body part in sections, but rest against the body part substantially completely or completely flat. Preferably, the first portion of the guiding/supporting structure encompasses a solid material or a combination of a plurality of materials, in particular carbon, PE, PP etc., which preferably gives the guiding/supporting structure its stability. In the state, in which it is coupled to the body part, however, this material or this material combination, respectively, is preferably not in contact with the body part or with the surface of the body part, respectively. Furthermore, the first portion of the guiding/supporting structure preferably encompasses a soft material or a combination of materials, in particular foam, textiles, rubber, etc., which are softer than the solid materials and which can preferably be brought into contact with the surface of the body part at least indirectly and preferably directly.

Such a guiding/supporting structure, which is preferably embodied as orthosis, has significant advantages as compared to the orthoses, which are known from the state of the art. Among others, the supporting/guiding structure according to the invention can thus in particular be used for preventing injuries in the field of sports, because, due to the adaptation according to the invention to the corresponding body part or to the corresponding body parts, respectively, no constrictions or significantly slighter or negligible constrictions, respectively, of the body part or of the body parts, respectively, occur as compared to the orthoses known from the state of art, in particular in the coupling area, that is, between the guiding/supporting structure and the body surface, in the case of different load states, in particular muscle tensioning states. The guiding/supporting structure according to the invention thus preferably serves to ergonomically absorb forces or to transfer forces, moments and/or movements, respectively, from the body of a living being, in particular from a human body, to the guiding/supporting structure placed against the body and/or vice versa. The guiding/supporting structure according to the invention can thus be used considerably more comfortably than the known orthoses for preventing injuries in response to increased loads, such as, e.g., in mass sports or even in competitive sports, or for preventing accidents, in particular in response to long, that is, several hours or permanent assignments or as part of an exoskeleton or in the field of rehabilitation devices, in particular for defined orientation of two or a plurality of body parts relative to one another or for the orientation of two or a plurality of parts of a body part, respectively, such as, e.g., when splinting a broken bone.

Among others, the guiding/supporting structure can be capable of being fixed to the respective body part or to the body parts, respectively, and/or to or on, respectively, or in clothing, respectively, e.g. by means of belts, in particular elastic or flexible belts, respectively, lacing, in particular elastic or flexible lacing, respectively. In the event that the guiding/supporting structure is integrated into the clothing, the guiding/supporting structure is preferably connected to the clothing in a non-detachable or detachable manner. Preferably, the guiding/supporting structure can be stuck or is stuck, respectively, to the clothing. The clothing can be tight-fitting, elastic clothing, e.g.

The flat portions, which can be brought into contact with the surface of the body part indirectly or directly, respectively, in particular the soft components of the guiding/supporting structure, are preferably placed against the further components, in particular the solid components, preferably in a form closure manner, by means of material connection, in a frictionally engaged manner and/or by means of a magnetic field, such as, e.g., adhered, sewn, riveted, welded, clamped, screwed, manufactured in an integrated manner, laminated and/or by means of a combination thereof. The flat portion or the flat portions, respectively, of the guiding/supporting structure for resting against the body parts, can also be identified as body-adapted surfaces, which can preferably be designed/or manufactured for being brought into contact with any body regions, parts, locations and limbs.

According to a further preferred embodiment of the present invention, the flat section is designed such that it can mainly or exclusively or substantially completely, respectively, be brought into contact with surface areas of the body part, which deform slighter than the further surface areas of the body part in response to a transition from a first load state into a second load state.

Preferably, the surfaces of the guiding/supporting structure thus surround the body or the body part, respectively, in an advantageous manner, without limiting the mobility or muscle volume change or surface deformation, respectively, of the body part, in particular not in an unintentional manner, and furthermore minimize the relative movement between the guiding/supporting structure and the body or the body part, respectively, or the body parts, respectively.

According to a further preferred embodiment, the second portion of the guiding/supporting structure is designed such that it can be placed against a further body part, in particular against a lower leg, such that in the defined orientation, which is provided for fulfilling the function of the guiding/supporting structure, with respect to the body part, in particular a thigh, it can be placed against surface areas of the further body part, which, when transitioning from a first load state into a second load state, encompass surface deformations, which are slighter as compared to the surface deformations of further surface areas of the further body part, during the same transition or which can be placed against surface areas of the further body part, which, when transitioning from a first load state into a second load state, encompass surface deformations, which are higher as compared to surface deformations of further surface areas of the further body part during the same transition.

This embodiment is advantageous, because the guiding/supporting structure, in particular as orthosis, can be coupled and/or inserted comfortably at a plurality of parts of a body part and/or at a plurality of body parts of a living being, in particular of a person.

According to a further preferred embodiment of the present invention, the shape of the flat section can be determined as a function of a method according to one of claims 1 to 6 or a production of the guiding/supporting structure is carried out as a function of a method according to one of claims 1 to 6, respectively.

Preferably, the determination or design, respectively, of the shape of the flat section as a function of the method according to the invention ensures that the guiding/supporting structure, which is needed or required, respectively, for the respective living being or the respective area of application, respectively, matches the living being optimally, that is, completely individually, and that the risk of constrictions and/or possible incorrect loading is reduced in an extremely advantageous manner.

According to a further preferred embodiment of the present invention, the shape of the flat section can be determined as a function of local surface deformation data, which can be assigned to groups of persons and/or groups of body parts and which are stored in a data bank and which can be determined according to one of claims 1 to 6.

This embodiment is advantageous, because guiding/supporting structures, which are not individually adapted to each living being, but which fit almost optimally for preferably defined groups, such as, e.g., men, skiers, leg length between 100 cm and 110 cm, can be designed and/or manufactured, e.g. according to a topology or surface structure, respectively, or surface characteristic, respectively, or surface shape, respectively, or surface deformation distribution, respectively, which is derived from serial measurements, in a highly time-consuming manner and thus relatively cost-efficiently.

According to a further preferred embodiment of the present invention, the flat section forms two partial areas, of which the surface area of the body part can at least be surrounded partially and between which a recess is embodied, wherein, in response to the transition from a first load state into a second load state, a surface area, which can be assigned to the recess, encompasses larger surface deformations than the surface areas, which can be overlaid by the flat section, or the recess is designed such, respectively, that the musculature of a living being in a tensioned state extends into the recess or extends between the flat sections, respectively, or extends further between the flat sections than is possible in an unloaded state, or a surface area, which can be assigned to the recess, encompasses slighter surface deformations than the surface areas, which can be overlaid by the flat section, in response to the transition from a first load state into a second load state.

This embodiment is advantageous, because the guiding/supporting structure can be coupled to the body part, preferably in a form closure manner, due to the embodiment of said guiding/supporting structure, which at least partially encloses a body part. Particularly preferably, the flat sections are thus designed such that, when used correctly in a provided application, they rest against surfaces with the smallest volume change of the body part or with the smallest contact shape changes of the surface of the body part, respectively, during a certain movement or activity. The recess furthermore forms an area, which allows for surface deformations of the body part, without resulting in constrictions.

According to a further preferred embodiment, the two partial areas of the flat section are connected to one another by means of a component, which extends in the area of the recess, in particular an at least partially elastic and/or flexible component. Preferably, the component thus tends to be shape-changeable. The component can thereby be a tape, a net, a textile, a membrane and/or combination thereof, e.g. The use of an elastic component is advantageous, because the overall stability of the guiding/supporting structure is increased, the component does not act in a constricting manner or only insignificantly constricts the body party of the living being and/or a shifting of the guiding/supporting structure is prevented or reduced, respectively, or hindered, respectively, further by means of the elastic component.

In the context of a further preferred embodiment, it is furthermore possible for the guiding/supporting structure to be an integral part of a piece of clothing, in particular of a pair of pants, of a T-shirt, a sweatshirt, a jacket, etc.

It is furthermore possible for the invention to also refer to a method for producing guiding/supporting structures, which can be coupled to a living being, wherein a detection of surface deformations of body surfaces of living beings takes place for determining suitable contact surfaces for guiding/supporting structures, which can be brought into contact with the surface of the living being or it is possible for the invention to refer to a method for producing a device for fastening different elements to the human body, respectively.

The production method or the method, respectively, preferably comprises at least the steps: at least one body part of the living being with respect to a detection device, in particular a 3-D scanner, at least partially detecting the surface deformations of at least one surface area of the body part, detecting a first surface characteristic or shape, respectively, of the surface area by means of the detection device, wherein the body part is in a first load state, in particular a state with the muscles relaxed, detecting a second surface characteristic or shape, respectively, of the surface area by means of the detection device, wherein the body part is in a second load state, which differs from the first load state, in particular a state with the muscles tensioned, wherein, in response to the detection in the various load states, it is possible for external influences to be considered and/or for the detection to be carried out with or without a contact to possible devices, and determining the local surface deformations resulting between the first load state and the second load state, wherein in particular a 3-D comparison of the body surface of the difference scans is carried out.

In further steps, the surfaces or the location shifts, respectively, which are suitable for optimally adapting a guiding/supporting structure to a living being, are additionally or alternatively extracted and, particularly preferably, are adapted to specific basic conditions or tasks, respectively, such as, e.g. load scenarios. In the case of an orthoses, it is possible that forces can be absorbed or need to be absorbed, respectively, so as to limit, avoid or influence bunions or internal rotary movements, e.g., for the purpose of which the coupling surfaces or the flat section of the first portion, respectively, needs to be adapted accordingly in design and/or with regard to production.

Preferably, a production control is carried out based on the 3-D data or CAD data, respectively, wherein it is also possible for the data to be able to serve as template for a manual production. The 3-D scans of the application-specific body regions cannot only be detected in different body positions or body postures, respectively, in particular comprising different load states, but also during a movement or can be extracted from a detected motion sequence, respectively. Preferably, at least two or exactly two scans are carried out, namely an initial and an end scan of the movement amplitude, wherein preferably a further or further, in particular exactly two or more than two, in particular exactly three or more than three, in particular exactly four or more than four, scans are made between the initial and end position.

Depending on the function of the guiding/supporting structure, a reference system is preferably defined or determined, respectively, in accordance with an additional or alternative method step. Particularly preferably, a 3-D comparison of the surface deformations or indirectly or directly a volume comparison of the body parts in the various load states is carried out in a further step, wherein a definition of the location shifts is preferably made. This can take place using the reference system or with regard thereto, respectively.

A production of such a guiding/supporting structure preferably takes place by means of a generative method and/ur using an additive production, wherein laminating tools and/or tools for sewing, knitting, weaving, etc., are preferably used in the production process as a whole. It is also possible for a tool, which preferably remains in the later guiding/supporting structure, to be generated in an additional or alternative method step, in particular in a production method step. Preferably, a production method is used or applied, respectively, in the case of which the 3-D data serve either directly or in a processed manner as base for the production control. It is furthermore possible for provision to be made for connecting elements as further attachment parts for connection to the body of the living being, in particular the human body. These connecting elements preferably serve to couple or to connect, respectively, or to guide the guiding/supporting structure to the body part or to the body parts, respectively, and/or to connect to further elements, which can be coupled to the living being via the guiding/supporting structure. Preferably, these connecting elements are embodied as belts, in particular as climbing harnesses, flexible climbing harnesses, leather belts, textile belts, tissue belts, or as lacing elements. The connecting elements are intentionally arranged or can be arranged, respectively, in rear areas of the guiding/supporting structure, so that an application of force can preferably take place on the rear side of the body part or on a side, which is located opposite a connecting means, respectively, for fixing the guiding/supporting structure to the body or to the body part, respectively, in a frictionally engaged and/or form closure manner.

Further advantages, goals and characteristics of the present invention will be explained by means of the description below of attached drawings, in which guiding/supporting structures according to the invention are illustrated in an exemplary manner. Components of the guiding/supporting structure according to the invention, which correspond substantially with regard to their function in the figures, can hereby be identified with the same reference numerals, wherein these components do not need to be numbered or explained in all of the figures.

Individual or all of the illustrations of the figures described hereinbelow are to preferably be considered as design drawings, that is, the dimensions, proportions, functional contexts and/or arrangements resulting from the figure or the figures, respectively, preferably correspond exactly or preferably substantially to those of the device according to the invention or of the product according to the invention, respectively.

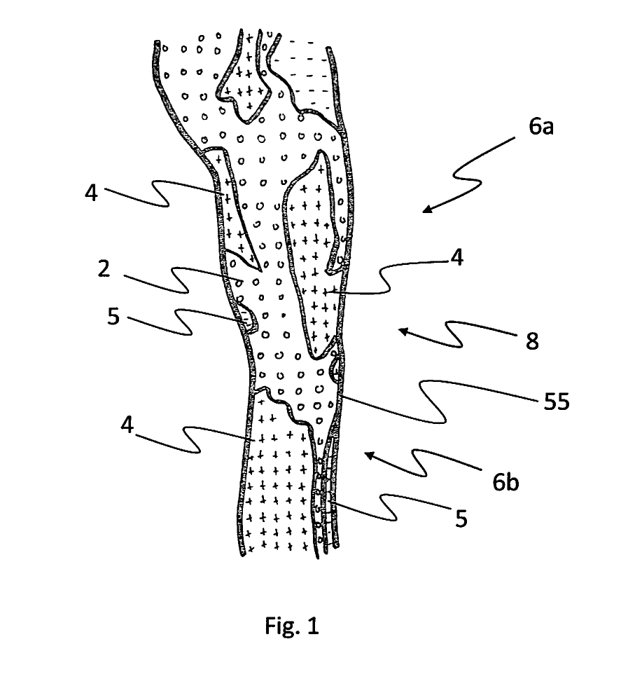

FIG. 1 shows an illustration of areas comprising different surface deformations of a body part of a person determined by means of the method according to the invention;

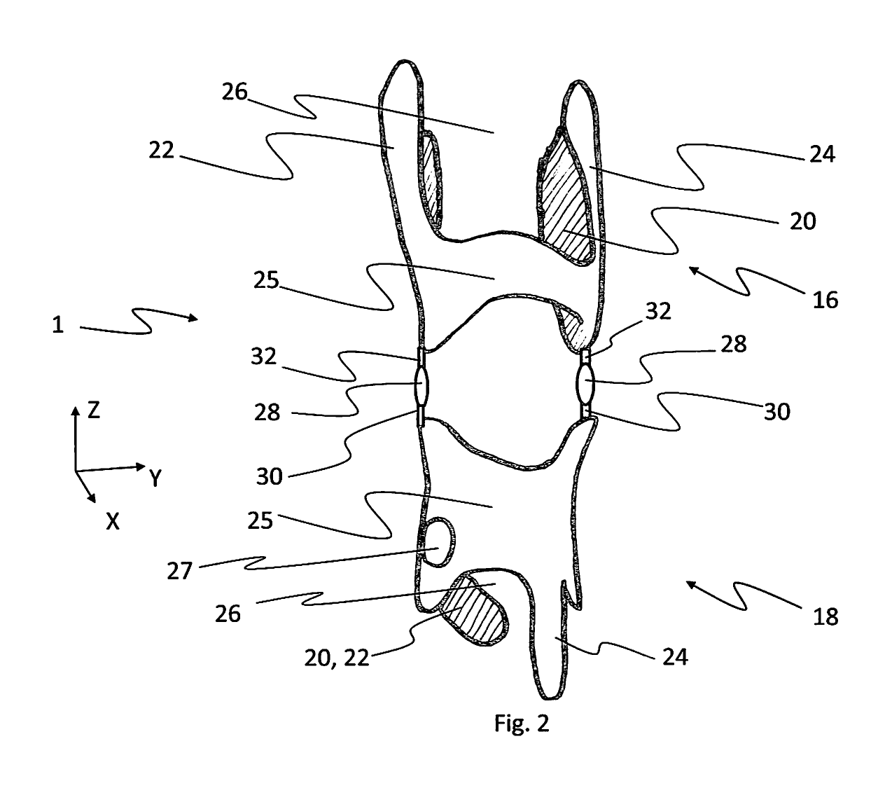

FIG. 2 shows a schematic illustration of a guiding/supporting structure, which is designed in consideration of the areas comprising a different surface deformation shown in FIG. 1;

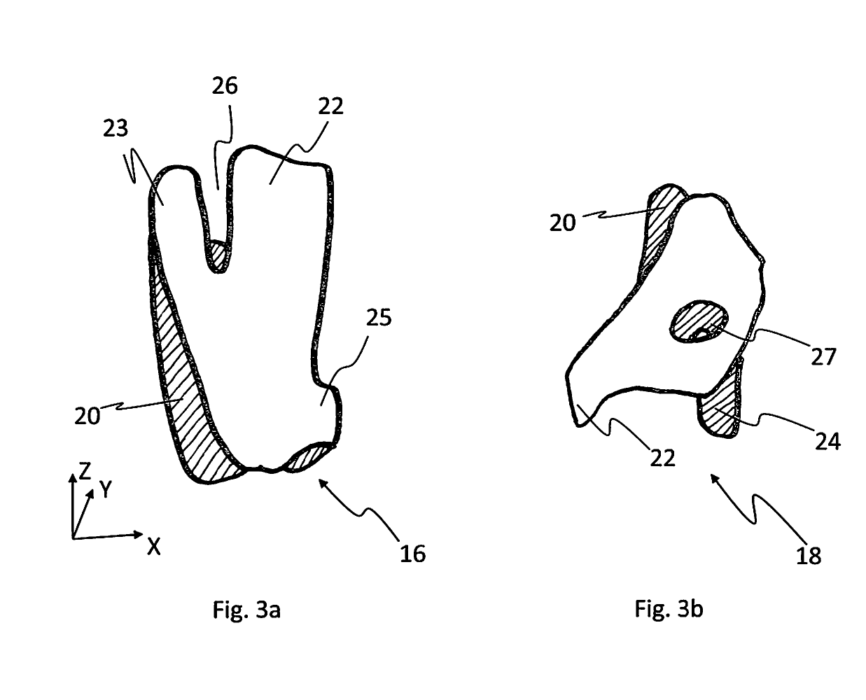

FIG. 3a shows a schematic lateral illustration of the upper portion of the guiding/supporting structure shown in FIG. 2;

FIG. 3b shows a schematic lateral illustration of the lower portion of the guiding/supporting structure shown in FIG. 2;

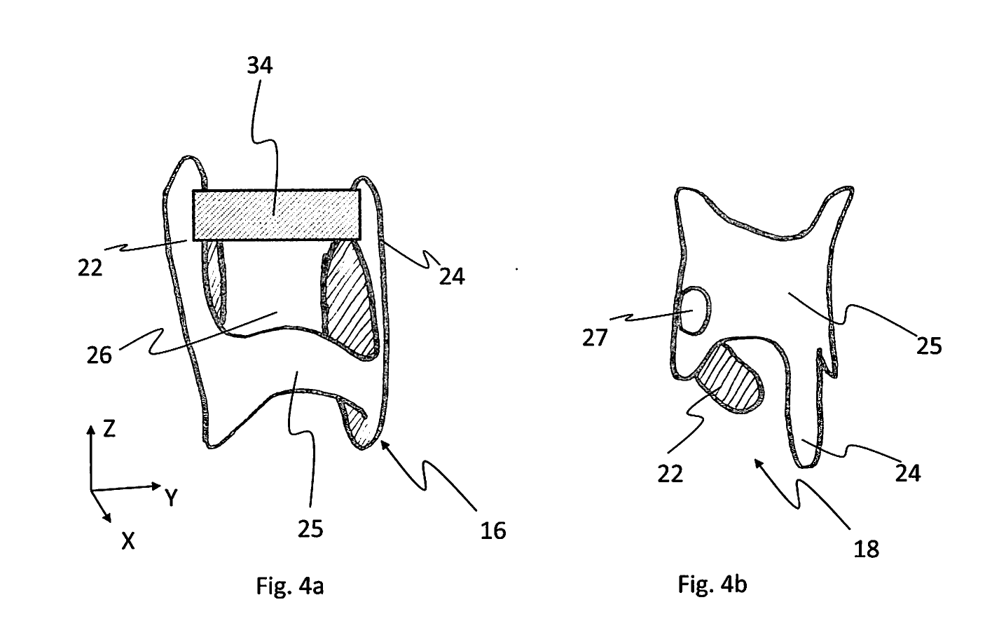

FIG. 4a shows a schematic frontal illustration of the upper portion of the guiding/supporting structure shown in FIG. 2, wherein a preferably flexible and/or elastic component is also shown;

FIG. 4b shows a schematic frontal illustration of the lower portion of the guiding/supporting structure shown in FIG. 2;

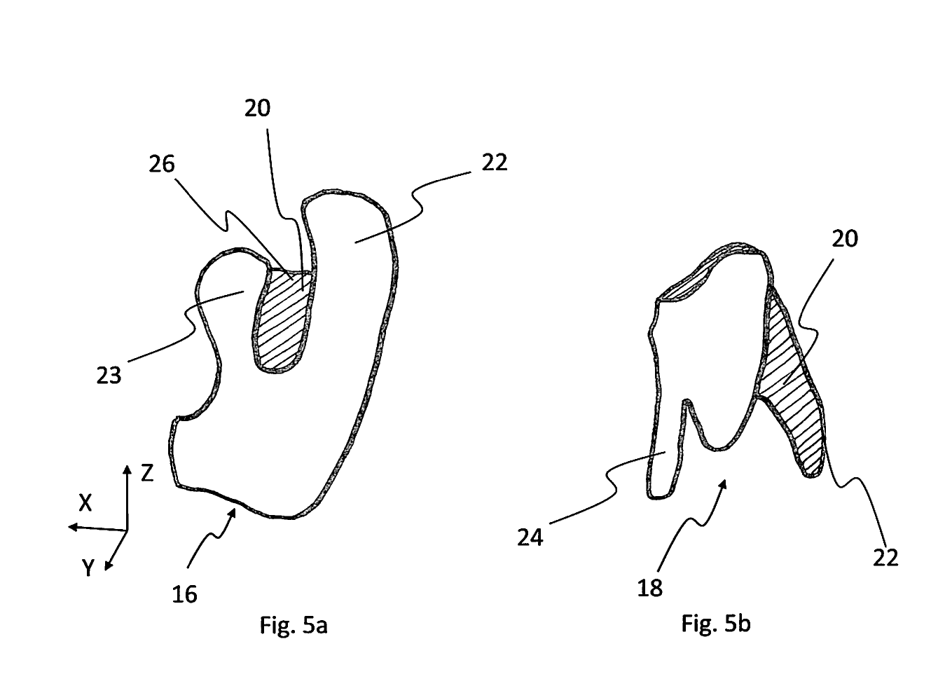

FIG. 5a shows a schematic medial illustration of the upper portion of the guiding/supporting structure shown in FIG. 2;

FIG. 5b shows a schematic medial illustration of the lower portion of the guiding/supporting structure shown in FIG. 2; and

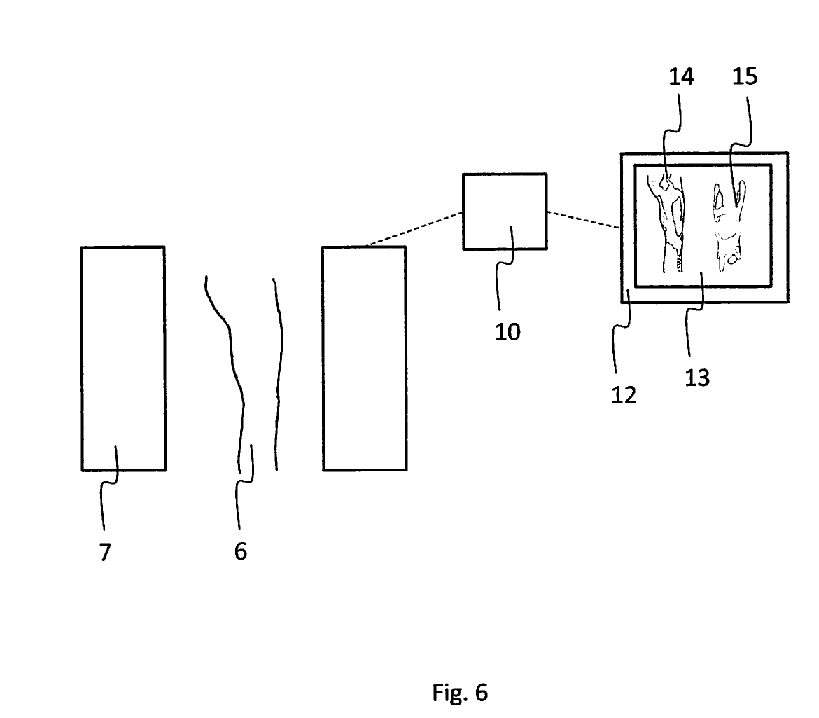

FIG. 6 shows an example of a device arrangement, by means of which the method according to the invention can be carried out.

FIG. 1 is an illustration of a body part 6 (6a, 6b). The body part 6 is a human leg and preferably encompasses at least partially a thigh 6a and a lower leg 6b. The surface area 8, which in this illustration comprises almost the entire thigh 6a and more than half of the lower leg 6b of the leg 6, is divided into different zones. In the example at hand, exactly three zone types 2, 4, 5, which differ from one another, are shown, wherein reference numeral 2 identifies a zone comprising slight volume changes or a body surface, which deforms slightly, respectively, and reference numerals 4, 5 identify zones comprising large volume changes or highly-deforming body surfaces, respectively. The surface deformations in the first zone 2, which can be assigned to the first surface area, are preferably slighter than the surface deformations in the second zone 4, which can be assigned to the second surface area, and preferably slighter than the surface deformations in third zone 5, which can be assigned to the third surface area, wherein the surface deformations in the third zone 5 with reference to a center of the body part occur in opposite direction to the surface deformations in the second zone 4. The third zone 5 can thus identify a zone, in which the surface deformation occurs due to a volume increase and the second zone 4 can identify a zone, in which the surface deformation results due to a volume increase. Preferably, the first zone 2 is a zone, in which no or substantially no surface deformation occurs, respectively.

Reference numeral 55 identifies a further zone. The further zone 55 preferably represents areas, in which no or only slight deformations occur, respectively, but which are considered as not being suitable for coupling to a guiding/supporting structure 1, due to the fixed parts, such as, e.g., bones or cartilage or implants, which are located very closely below the surface. Data relating to the position of such zones 55 can preferably be provided and/or can be recalled from a data storage or a data library, respectively, and/or can be identified at the body itself prior to the surface detection.

FIG. 2 shows a schematic perspective illustration of a preferred guiding/supporting structure 1, in particular of a leg or knee orthosis. The orthosis 1 encompasses an upper portion 16 and a lower portion 18. Preferably, the upper portion 16 is embodied such that it can be coupled to the thigh 6a of the leg shown in FIG. 1 in the manner according to the invention. According to the detected surface areas 2 and/or 4 and/or 5, the lower portion 18 of the orthosis 1 is also embodied, that is, it is designed such that it can be coupled to the lower leg 6b shown in FIG. 1 in the manner according to the invention. The upper portion 16 and the lower portion 18 are preferably connected to one another via one or a plurality of flexible connecting pieces 28, respectively, and particularly preferably via a joint 28 or two joints 28, respectively. The joint or the joints 28, respectively, can thereby be arranged or embodied, respectively, directly at the upper portion 16 and/or at the lower portion 18, wherein they are preferably arranged at the first and second portion 16, 18 by means of lower connections 30 and/or upper connections 32.

The upper portion 16 preferably encompasses at least a first partial area 22 and a second partial area 24 for lateral or bilateral enclosing the thigh 6a, respectively, whereby the upper portion 16 can preferably be coupled to the thigh 6a in a form closure manner. The two partial areas 22, 24 preferably extend in the longitudinal direction of the leg 6 or of the thigh 6a, respectively, and are preferably connected to one another by means of a connecting means 25, which preferably extends in circumferential direction of the leg. Particularly preferably, the partial areas 22, 24 and possible further partial areas 23 (see FIG. 3a) as well as the connecting means 25 together form a unit, which is created in one piece. However, it is also possible for some of these elements 22, 23, 24, 25 or all of the elements 22, 23, 24, 25 to be releasably connected or replaceably connected to one another, respectively. According to this illustration, the partial areas 22, 24 define a recess 26. The recess 26 represents an area, in which the body part 6 or the thigh 6a, respectively, can be subject to larger volume changes or surface deformations, respectively, in particular expansions and contractions as compared to the areas, which are overlaid by the partial areas 22, 24, without resulting in a constriction of the body part 6 in response to a volume change of the body part 6 or in response to a deformation of the body part surface 4, respectively. On the inner side, at least a partial area 22, 23, 24 and preferably the first partial area 22 and/or the second partial area 24 and particularly preferably the first partial area 22, the second partial area 24 and exactly a further partial area 23 or at least a further partial area 23 are embodied as a flat section 20 such that it can be placed against or arranged, respectively, against the surface of the body part 6 at least flat in sections and at least indirectly, in particular against surface areas 2, which are only subject to slight or no, respectively, or substantially no volume changes or surface deformations, respectively, between two load states. It is also possible for the connecting element 25, which preferably extends in circumferential direction of a body part 6, to also be designed for resting against the body surface 2 or for being brought into contact with the body surface 2 on the inner side, respectively. If the connecting means 25 is arranged or provided, respectively, in the area of a highly deforming body surface 4, it is preferably designed such that it cannot be brought into contact with the body surface 4 or only insignificantly, respectively.

The above-described characteristics can preferably be applied analogously with reference to the elements 20, 22, 24, 25 of the second section 18. It can further be gathered from the illustration that the first partial area 22 of the second section 18 extends in a different direction than the second partial area 24 of the second section 18. The longitudinal axis of the first partial area 22 preferably extends in longitudinal direction of the guiding/supporting structure 1 and in circumferential direction of the guiding/supporting structure 1, whereas the second partial area 24 extends substantially in the longitudinal direction Z of the guiding/supporting structure 1 or only in the longitudinal direction Z, respectively, of the guiding/supporting structure 1. It is nonetheless also possible for the longitudinal axes of both partial areas 22, 24 to extend at least partially in circumferential direction in a further embodiment or to extend parallel or substantially parallel, respectively, to the longitudinal axis Z of the guiding/supporting structure 1.

It can furthermore be gathered from the illustration that the connecting means 25 of the upper section 16 encompasses a slighter expansion or extension, respectively, in the longitudinal direction of the guiding/supporting structure 1 than the connecting means 25 of the second section 18. However, it is also possible for this ratio to be reversed or for both connecting means 25 to extend substantially across the same distance or exactly the same distance in the longitudinal direction of the guiding/supporting structure 1. The partial areas 22 and 24 of the second section 18 preferably also define a recess 26, which is designed for defining or limiting, respectively, deformations of the body part surface 4 of a living being as little as possible. It can be gathered from this illustration that the recess 26 of the second section 18 is embodied so as to be offset or rotated, respectively, with respect to the recess 26 of the first section 16 in circumferential direction of the guiding/supporting structure 1. Depending on the field of application, living beings, etc., it is possible for the recesses 26 of the first and second section 16, 18 to rotate or to be oriented substantially aligned or exactly aligned to one another, respectively.

It can furthermore be gathered from the illustration that both the upper as well as the lower section 16, 18 preferably embody two or exactly two, respectively, or at least two recesses 26 in each case. Preferably, the number of the recesses 16 is a function of the number of partial areas 22, 23, 24, which can preferably differ, depending on the field of application, living being and/or body part, etc.

Reference numeral 27 identifies a further recess, in particular a hole. The shape of the recess 27 can thereby encompass straight portions and/or spherical portions, wherein it is also possible that it only encompasses straight portions or only spherical portions. The recess 27 can be embodied to reduce the weight, to relieve tension and/or to release surface areas 4, in particular to provide arbitrary surface deformations in the released or non-overlayable surface area 4, respectively.

FIG. 3a shows an illustration of the upper section 16 in a lateral orientation. A further partial area 23 can be seen thereby, which, together with the first partial area 22, defines a recess 26. The connecting area 25 is furthermore embodied such that it preferably extends at right angles from the center or the longitudinal direction, respectively, of the guiding/supporting structure 1 to the outside. This embodiment provides the advantage that the connecting means 25 overlays the body part 6 of the living being at such a distance that it cannot be brought into contact with the body surface 4. Preferably, the areas, which are hatched, are flat sections 20, which are designed to rest against the living being.

FIG. 3b shows an illustration of the lower section 16 in a lateral orientation. The orientation of the first partial area 22 of the lower section 18, in particular the partial extension thereof in longitudinal direction and circumferential direction, can be seen by means of this illustration. The preferably round or oval, respectively, or substantially round, respectively, or substantially oval shape of the further recess 27 can furthermore be seen. It is furthermore possible for further recesses 27, in particular holes, to be embodied in addition or alternatively in the second section 18 and/or in the first section 16.

FIG. 4a shows an illustration of the upper section 16 in a frontal orientation. According to this illustration, the first partial area 22 and the second partial area 24 of the first section 16 are connected to one another by means of an elastic component 34, which can be embodied as belt, tape, tissue, etc. The elastic component 34 can thereby be arranged or embodied at the upper part or in the area of the upper end of the guiding/supporting structure 1, respectively, wherein it is also possible for it to additionally or alternatively be arranged or embodied in the central area of the recess 26 or in the lower area of the recess 26, respectively. It is furthermore possible for the elastic component 34 to extend substantially across the entire recess 26 or to extend exactly across the entire recess 26. Preferably, the elastic component 34 is arranged at the guiding/supporting structure 1, in particular at two partial areas 22, 23, 24 so as to be capable of being detached, in particular by means of a form closure locking, in a frictionally engaged manner and/or by means of a magnetic field, in particular by means of a hook-and-loop fastener, zipper, snap fasteners, etc. However, it is also possible for the elastic component 34 to be connected to the guiding/supporting structure 1 in a fixed manner, that is, preferably in a non-destructive manner. Furthermore, it is additionally or alternatively possible for one or a plurality of elastic components 34 to be arranged on the rear side of the supporting/guiding structure 1 as connecting elements for coupling the guiding/supporting structure 1 to the body part 6a or the body 6, respectively.

FIG. 4b shows a further illustration of the lower section 16 in a frontal orientation.

FIG. 5a shows an illustration of the upper section 16 in a medial orientation. It can be gathered from this illustration that e.g. the further partial area 23 encompasses a spherical outer contour. This design results from a preferably exact analysis of the surface areas 2, 4, 5, which are shown in FIG. 1, so that a surface, which is as large as possible, of the slightly deforming surface areas 2 can be used for coupling and thus for transferring forces, because larger forces can be transferred as the surface increases or an even higher comfort for the living being can be generated in response to constant forces, respectively.

FIG. 5b shows a further illustration of the lower section 16 in a medial orientation. The partial areas 22, 23, 24 shown in FIG. 5b are preferably of different lengths, different widths and are preferably designed with a different outer contour in each case, wherein it is possible in a particularly preferred manner for the partial areas 22, 23, 24 to correspond in sections or completely at least partially or completely in length. In a particularly preferred manner, the partial areas 22, 23, 24 can thus be designed as a function of the respective application, of the respective living being, etc.

FIG. 6 shows an arrangement, by means of which the method according to the invention is carried out in a preferable manner. However, it is possible hereby for additional or alternative mechanisms, devices, means and/or methods to be used. The body part 6 is located in the area of a detection device 7, which surrounds the body part 6 at least temporarily in sections and preferably completely. The deformation values detected by the detection device 7 are supplied to a processor device 10, which is preferably connected to the detection device 7 at least in terms of signaling. The processor device 10 determines or computes, respectively, the deformations, which occur when the body part 6 of the living being is examined and/or scanned and/or detected or measured, respectively, in different load states.

Preferably, the results generated by the processor device 10 can be output via a display device 12, which is preferably embodied as screen 13. Among others, a 3-D model 14 of the body part 6 and/or a model 15, which is generated by means of the 3-D model 14, of a guiding/supporting structure 1 according to the invention, can be gathered at the same time or successively on the screen 13 in accordance with the explanations relating to FIG. 1.

### LIST OF REFERENCE NUMERALS

1 guiding/supporting structure

2 slightly deforming body surface

4 highly deforming body surface

5 highly deforming body surface, in particular inverse to the body surface 4

6a first body part

6b further body part

7 detection device

8 surface area

10 processor device

12 display device

13 screen

14 3-D model

15 model of the supporting/guiding structure

16 first portion

18 second portion

20 flat section

22 first partial area

23 further partial area

24 second partial area

25 connecting element

26 recess

27 further recess

28 joint

30 bottom-side connection

32 upper-side connection

34 elastic component

55 further zone

X depth

Y width

Z length

1. A method for detecting surface deformations of a body surface of a living being for determining suitable contact surfaces for guiding/supporting structures, which can be brought into contact with the surface of the living being, the method comprising:
orienting a body part of the living being with respect to a detection device for at least partially detecting a surface shape of a surface area of the body part,
detecting a first surface shape of the surface area by means of the detection device when the body part is in a first load state,
detecting a second surface shape of the surface area by means of the detection device when the body part is in a second load state, which differs from the first load state,
determining local surface deformations resulting between the first load state and the second load state.

2. The method according to claim 1 wherein
a comparison of the detected local surface deformations is carried out with a defined threshold value or a defined threshold value function in response to determining the local surface deformations, wherein deformation values resulting therefrom can be described on one side of the defined threshold value or the defined threshold value function as values having a slight surface deformation, and can be described on the other side of the defined threshold value or of the defined threshold value function as values of a high surface deformation.

3. The method according to claim 2 wherein
the determination of the local surface deformations and/or the comparison of the detected surface deformations is effected by means of a processor device.

4. The method according to claim 2 wherein
an illustration, which corresponds to the deformation values and/or to surface areas, which can be assigned on the one side of the threshold value or the threshold value function, is generated and/or visualized for displaying different degrees of deformation at different positions of the surface area.

5. The method according to claim 2, wherein
the deformation values and/or surface areas, which can be assigned on the one side of the threshold value or of the threshold value function, are transferred into data for generating a 3-D model.

6. A computer-implemented method according to claim 1.

7. A guiding/supporting structure for coupling to a living being for transferring forces, which can be introduced into a first portion of the guiding/supporting structure, to a second portion of the guiding/supporting structure, which is spaced apart from the first portion,
wherein the first portion is embodied for resting against a surface area of a body part of a living being, at least indirectly and at least with a flat section,
wherein at least the flat section of the first portion is embodied for resting against the body part such that
in a defined orientation with respect to the body part, which is provided for fulfilling the function of the guiding/supporting structure, the flat section can be placed against surface areas, which, when transitioning from a first load state into a second load state, encompass surface deformations, which, during the same transition, are slighter than surface deformations of further surface areas of the body part, or,
in a defined orientation with respect to the body part, which is provided for fulfilling the function of the guiding/supporting structure, the flat section can be placed against surface areas, which, when transitioning from a first load state into a second load state, encompass surface deformations, which, during the same transition, are higher than surface deformations of further surface areas of the body part.

8. The guiding/supporting structure according to claim 7 wherein
at least three surface areas are assigned to zones of different surface deformations as a function of at least two defined threshold values or threshold value functions.

9. The guiding/supporting structure according to claim 7 wherein
the flat section is designed such that it can mainly be brought into contact with surface areas of the body part, which deform slighter than the further surface areas of the body part in response to a transition from a first load state into a second load state.

10. The guiding/supporting structure according to claim 7 wherein
the flat section is designed such that it can exclusively or substantially exclusively be brought into contact with surface areas of the body part, which deform slighter than the further surface areas of the body part in response to a transition from a first load state into a second load state.

11. The guiding/supporting structure according to claim 7 wherein
the second portion of the guiding/supporting structure can be placed against a further body part such that in the defined orientation, which is provided for fulfilling the function of the guiding/supporting structure with respect to the body part, the second portion can be placed against surface areas of the further body part, which, when transitioning from a first load state into a second load state, encompass surface deformations, which are slighter with respect to surface deformations of further surface areas of the further body part during the same transition, or,
can be placed against surface areas of the further body part, which, when transitioning from a first load state into a second load state, encompass surface deformations, which are higher with respect to surface deformations of further surface areas of the further body part during the same transition.

12. The guiding/supporting structure according to claim 7 wherein
shape of the flat section can be determined as a function of a method according to claim 1.

13. The guiding/supporting structure according to claim 7 wherein
shape of the flat section can be determined as a function of local surface deformation data, which can be assigned to groups of persons and/or groups of body parts and which are stored in a data bank and which can be determined according to the method of claim 1.

14. The guiding/supporting structure according to claim 7 wherein
the flat section forms two partial areas, of which the surface area of the body part can at least be surrounded partially and between which a recess is embodied, wherein, in response to the transition from a first load state into a second load state, a surface area, which can be assigned to the recess, encompasses larger surface deformations than the surface areas, which can be overlaid by the flat section, or
a surface area, which can be assigned to the recess, encompasses slighter surface deformations than the surface areas, which can be overlaid by the flat section, in response to the transition from a first load state into a second load state.

15. The guiding/supporting structure according to claim 14 wherein
the two partial areas of the flat section are connected by means of a component which extends in the area of the recess.

16. The method of claim 1 wherein the first load state is a state with muscles of the living being relaxed, and the second load state is a state with the muscles tensioned.

17. The guiding/supporting structure according to claim 8 wherein the surface deformations in the first zone, which can be assigned to the first surface area, are slighter than the surface deformations in the second zone, which can be assigned to the second surface area, and are slighter than the surface deformations in the third zone, which can be assigned to the third surface area, wherein the surface deformations in the third zone with reference to a center of the body part preferably take place in an opposite direction to the surface deformations in the second zone.
